(19) United States
(12) Patent Application Publication
(10) Pub. No.: US 2022/0015709 A1
(43) Pub. Date: Jan. 20, 2022

(54) KEYBOARD DEVICE HAVING FUNCTIONALITY OF PHYSIOLOGICAL PARAMETER MEASUREMENT

(71) Applicant: Primax Electronics Ltd., Taipei (TW)

(72) Inventors: Ling-Bo Li, Taipei (TW); Xiao-Ping Wang, Taipei (TW); Liu-Bing Cai, Taipei (TW)

(21) Appl. No.: 17/002,858

(22) Filed: Aug. 26, 2020

(30) Foreign Application Priority Data
Jul. 14, 2020 (CN) 202010673297.3

(51) Int. Cl. A61B 5/00; A61B 5/0205; G16H 40/67; G16H 50/30; G06F 3/02

(52) U.S. Cl. CPC A61B 5/6897; A61B 5/0205; A61B 5/7445; A61B 5/7425; A61B 5/021; A61B 5/7257; G16H 40/67; G16H 50/30; G06F 3/0219; A61B 5/0002

(57) ABSTRACT

A keyboard device having functionality of physiological parameter measurement is disclosed, which comprises a keyboard main body and at least two physiological signal sensing units. The physiological signal sensing unit comprises a lighting element and a light sensing element. Moreover, the physiological signal sensing unit further comprises a touch plane that is exposed out of the surface of the keyboard main body. When a user is typing the button keys of the keyboard main body, the user can touch the touch plane by one finger thereof, such that the physiological parameter calculating unit receives an optical signal through the physiological signal sensing unit, thereby calculating physiological parameters of the user after applying a physiological parameter calculating process to the optical signal. The calculated physiological parameter comprises heartbeat and heart rate.

### FIELD OF THE INVENTION

The present invention relates to the technology field of computer keyboard devices, and more particularly to a keyboard device having functionality of physiological parameter measurement.

### BACKGROUND OF THE INVENTION

Blood oxygen saturation level (SpO₂), heartbeat, heart rate (HR), and blood pressure are known important physiological parameters for judging a person's health states. Nowadays, photoplethysmography has been applied in measurement of the forgoing physiological parameters. For example, Taiwan Patent No. 1592138 discloses a wearable blood-pressure measuring apparatus, which is the current on-sale smart band for being worn on a user's wrist, thereby completing a measurement of a plurality of physiological parameters from the user's wrist by utilizing photoplenthysmography sensors. On the other hand, U.S. Patent Publication No. 2017/0340217A1 discloses a physiological detection device, which is a fingertip pulse oximeter. To measure blood-oxygen (SpO₂) from a user, it requires user to put his index finger into a finger receiving space of the fingertip pulse oximeter. After that, the fingertip pulse oximeter generates a detection light for illuminating the front surface of the index finger, and then a photoplethysmography (PPG) signal is measured by successively recoding a transmission light from the rear surface of the index finger.

It is known that both the smart band and the fingertip pulse oximeter are portable. However, user feedback report still indicates that the forgoing two physiological parameter measuring devices have drawbacks in practical use. For example, if a user has already gone out without wearing the smart band or carrying the fingertip pulse oximeter, the user cannot to record and manage his daily health states because of lacking one physiological parameter measuring device for completing a measurement of physiological parameters.

Nowadays, computer is the most important electronic device that is utilized for assisting people in completing their works quickly and effectively. Therefore, besides the smart phone and the tablet PC, computer keyboard is the electronic device that people spend most of time to use in every day. Thus, if one computer keyboard is provided with a functionality of physiological parameter measuring, a user is able to record and manage his daily health states by incidentally achieving a physiological parameter measurement during using the computer keyboard. Because of the above reasons, inventors of the present application have made great efforts to make inventive research and eventually provided a keyboard device having functionality of physiological parameter measurement.

### SUMMARY OF THE INVENTION

A primary objective of the present invention is to provide a keyboard device having functionality of physiological parameter measurement. The keyboard device comprises a keyboard main body and at least two physiological signal sensing units. In which, the keyboard main body has a main controller, and the main controller is provided with a physiological parameter calculating unit therein. According to the present invention, the at least two physiological signal sensing units are disposed in the keyboard main body, and each of the two physiological signal sensing units comprises a lighting element and a light sensing element. Moreover, the physiological signal sensing unit has a touch plane that is exposed out of the surface of the keyboard main body. By such arrangement, when a user is typing the key buttons of the keyboard main body, the user can touch the touch plane by one finger thereof, such that the physiological parameter calculating unit receives a first optical signal through the physiological signal sensing unit, thereby calculating physiological parameters of the user after applying a physiological parameter calculating process to the first optical signal. The calculated physiological parameter comprises heartbeat and heart rate.

Moreover, during typing the key buttons of the keyboard main body, the user can touch another one touch plane by another one finger thereof, such that the physiological parameter calculating unit receives a second optical signal through another one physiological signal sensing unit, thereby calculating physiological parameters of the user after applying the physiological parameter calculating process to the second optical signal. The calculated physiological parameter comprises heartbeat, heart rate, blood pressure, and blood oxygen saturation level.

To achieve the foregoing objective, the present invention provides one embodiment for the keyboard device having functionality of physiological parameter measurement, comprising:

a keyboard main body having a main controller and a plurality of key buttons, wherein the main controller is provided with a physiological parameter calculating unit therein; and

at least two physiological signal sensing units, being disposed in the keyboard main body; wherein each of the at least two physiological signal sensing units comprises at least one lighting element and at least one light sensing element, and further comprising a touch plane that is exposed out of a surface of the keyboard main body;

wherein in case of a first finger of a user pressing one physiological signal sensing unit via the touch plane, the lighting element emitting a first detection light to the first finger for making the light sensing element receive a first reflective light from the first finger, such that the main controller receives a first sensing signal from the light sensing element, so as to utilize the physiological parameter calculating unit to calculate at least one first physiological parameter after applying a physiological parameter calculating process to the first sensing signal;

wherein in case of a second finger of the user simultaneously pressing another one physiological signal sensing unit via the touch plane, the lighting element emitting a second detection light to the second finger for making the light sensing element receive a second reflective light from the second finger, such that the main controller receives a second sensing signal from the light sensing element, so as to utilize the physiological parameter calculating unit to calculate at least one second physiological parameter after applying the physiological parameter calculating process to the second sensing signal;

wherein the at least one first physiological parameter and the at least one second physiological parameter comprises heartbeat, heart rate, blood pressure, and blood oxygen saturation level.

In the embodiment of the forgoing keyboard device having functionality of physiological parameter measurement, the keyboard main body further comprises a display device that is coupled to the main controller, and a display screen of the display device being exposed out of the keyboard main body, such that the main controller shows an indicating information, through the display screen, for informing the user that a physiological parameter measurement is beginning to be executed.

In the embodiment of the forgoing keyboard device having functionality of physiological parameter measurement, the main controller shows the heartbeat, the heart rate, the blood pressure, and/or the blood oxygen saturation level on the display screen after the physiological parameter measurement is completed.

In the embodiment of the forgoing keyboard device having functionality of physiological parameter measurement, the main controller transmits a physiological parameter data comprising the heartbeat, the heart rate, the blood pressure, and the blood oxygen saturation level to an electronic device through a wired communication interface or a wireless communication interface, such that the electronic device shows the heartbeat, the heart rate, the blood pressure, and/or the blood oxygen saturation level on a display screen thereof.

In the embodiment of the forgoing keyboard device having functionality of physiological parameter measurement, the electronic device is selected from the group consisting of desk computer, laptop computer, tablet computer, smart phone, and smart TV.

In the embodiment of the forgoing keyboard device having functionality of physiological parameter measurement, the electronic device has a physiological parameter managing program for achieving a data management of the physiological parameter data, and the data management is selected from the group consisting of data storing, data transmitting, displaying the physiological parameter data by a form of graphs or diagrams, and displaying the physiological parameter data by a form of numeric values.

In the embodiment of the forgoing keyboard device having functionality of physiological parameter measurement, the lighting element is selected from the group consisting of light-emitting diode (LED), quantum dot light emitting diode (QD-LED), mini LED, micro LED, and organic light-emitting diode (OLED).

In the embodiment of the forgoing keyboard device having functionality of physiological parameter measurement, both the first detection light and the second detection light are a single-wavelength light or a multi-wavelength light.

### Organic Light-Emitting Diode (OLED).

In the embodiment of the forgoing keyboard device having functionality of physiological parameter measurement, the light sensing element is selected from the group consisting of single point photo sensor, matrix photo sensor, one-channel image sensor, and multi-channel image sensor.

In the embodiment of the forgoing keyboard device having functionality of physiological parameter measurement, both the first sensing signal and the second sensing signal are a photoplethysmography (PPG) signal, and the physiological parameter calculating unit applying at least one signal process to the first sensing signal and the second sensing signal before executing the physiological parameter calculating process.

In the embodiment of the forgoing keyboard device having functionality of physiological parameter measurement, the signal process is selected from the group consisting of fast Fourier transforming (FFT) process, discrete Fourier transforming (DFT) process and short-time Fourier transforming (STFT) process.

In the embodiment of the forgoing keyboard device having functionality of physiological parameter measurement, the signal process is selected from the group consisting of signal process using singular spectrum analysis (SSA) algorithm and signal process using normalized least mean square (NLMS) algorithm.

### BRIEF DESCRIPTION OF THE DRAWINGS

FIG. 1 shows a top-view diagram of a keyboard device having functionality of physiological parameter measurement according to the present invention.

FIG. 2 shows a block diagram of the keyboard device having functionality of physiological parameter measurement according to the present invention.

FIG. 3 shows a stereo diagram of an electronic device that is coupled to the keyboard device having functionality of physiological parameter measurement.

FIG. 4 shows a top-view diagram of a keyboard device having functionality of physiological parameter measurement according to the present invention.

FIG. 5 shows a block diagram of the keyboard device having functionality of physiological parameter measurement according to the present invention.

FIG. 6 shows a top-view diagram of a keyboard device having functionality of physiological parameter measurement according to the present invention.

FIG. 7 shows a block diagram of the keyboard device having functionality of physiological parameter measurement according to the present invention.

### DETAILED DESCRIPTION OF THE PREFERRED EMBODIMENT

The advantages and features of a keyboard device having functionality of physiological parameter measurement according to the present invention are described in details with reference to examples of embodiments and accompanying drawings to be more easily understood. However, the present invention may be implemented in different forms, and should not be construed as limited to only embodiments described herein. Conversely, for a person skilled in the art, the embodiments are provided for making the disclosure more thorough and comprehensive and completely conveying the scope of the present invention.

### First Embodiment

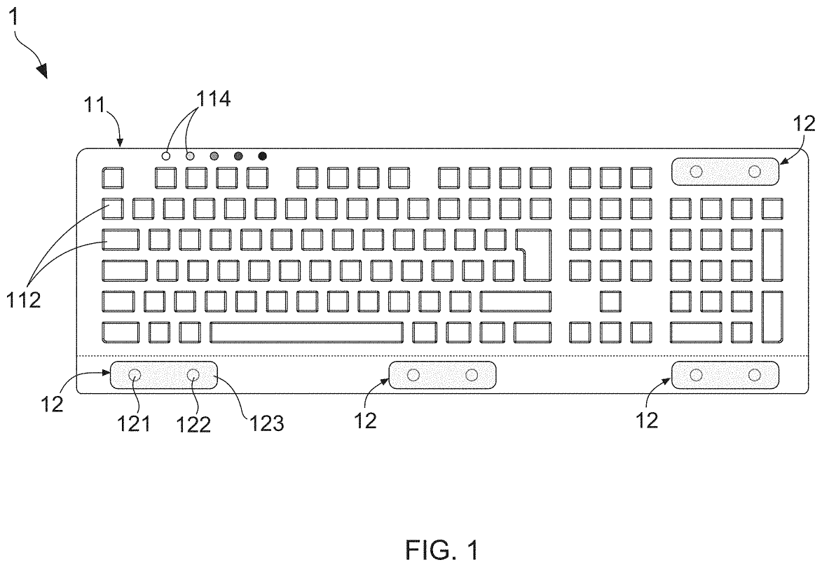

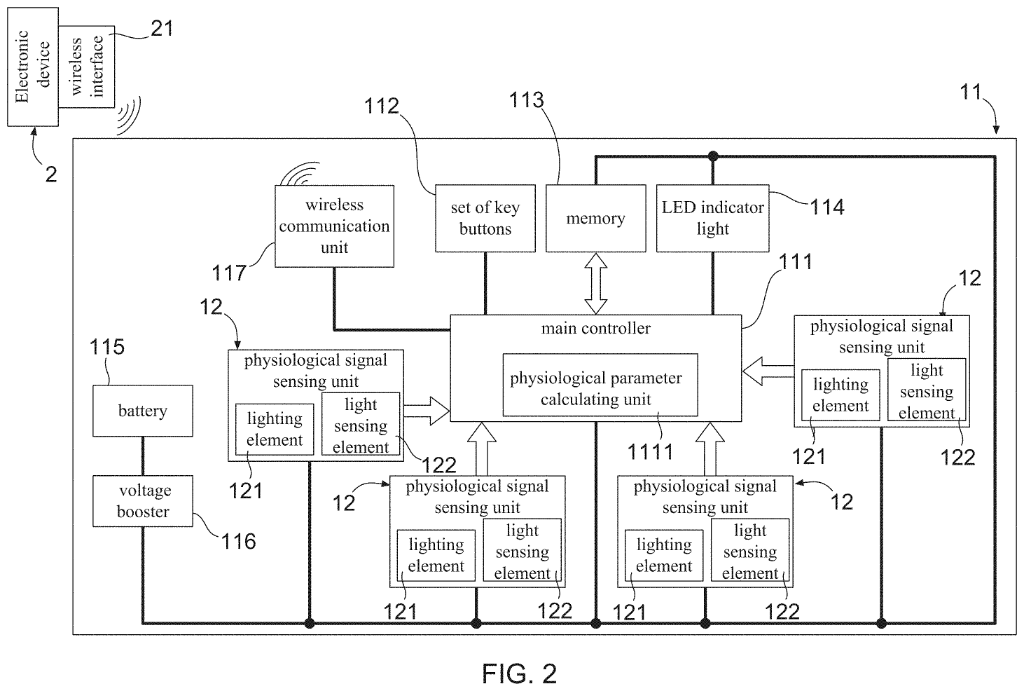

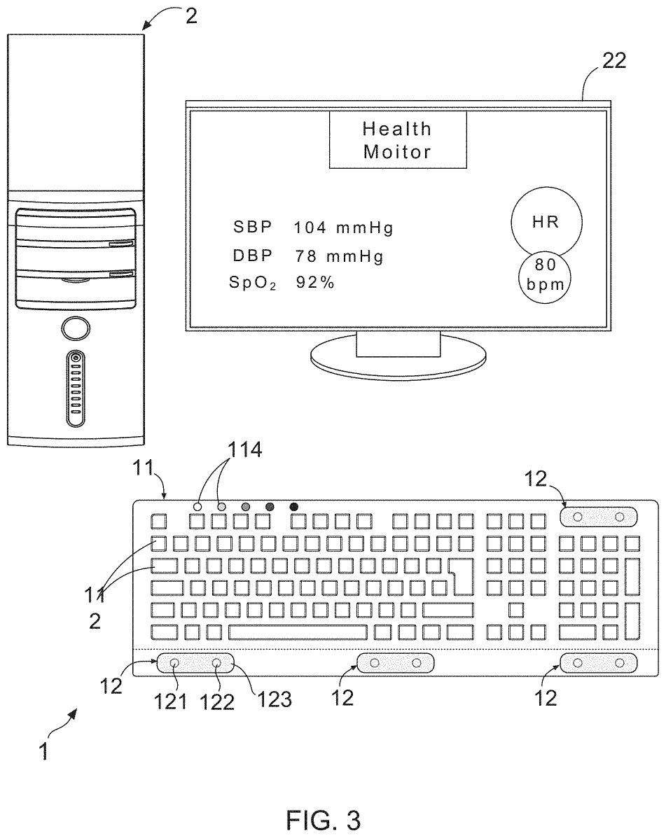

With reference to FIG. 1, there is provided a top-view diagram of a keyboard device having functionality of physiological parameter measurement according to the present invention. Moreover, FIG. 2 shows a block diagram of the keyboard device having functionality of physiological parameter measurement according to the present invention. As FIG. 1 and FIG. 2 show, the keyboard device 1 of the present invention comprises a keyboard main body 11 and at least two physiological signal sensing units 12. FIG. 3 illustrates a stereo diagram of an electronic device that is coupled to the keyboard device 1 of the present invention. In first embodiment, the keyboard main body 11 is a wireless computer keyboard, and principally comprises a main controller 111, a set 112 of key buttons, a memory 113, at least one LED indicator light 114, a battery 115, a voltage booster 116, and a wireless communication unit 117. Herein, wireless computer keyboard is a well-known inputting device, which communicates with a wireless interface 21 of an electronic device 2. For this reason, it is not needed to introduce the basic constituting elements of the wireless computer keyboard.

Particularly, the present invention lets a physiological parameter calculating unit 1111 be provided in the main controller 111 of the keyboard main body 11, and disposes at least two physiological signal sensing units 12 in the keyboard main body 11. As FIG. 1, FIG. 2 and FIG. 3 show, each of the physiological signal sensing units 12 comprises at least one lighting element 121 and at least one light sensing element 122, and further comprising a touch plane 123 that is exposed out of a surface of the keyboard main body 11. By such arrangement, in case of a first finger of a user pressing one physiological signal sensing unit 12 via the touch plane 123, the lighting element 121 emit a first detection light to the first finger for making the light sensing element 122 receive a first reflective light from the first finger, such that the main controller 111 receives a first sensing signal from the light sensing element 122, so as to utilize the physiological parameter calculating unit 111 to calculate physiological parameters of the user after applying a physiological parameter calculating process to the first sensing signal. Because the blood flow rate in the vessel would vary with the user's heartbeat, an amount of absorption of the first detection light that is absorbed by the blood flowing in the vessel would also change. As a result, after applying a physiological parameter calculating process to the first sensing signal, the physiological parameter calculating unit 1111 calculates the user's physiological parameters including heartbeat and heart rate.

Moreover, in case of a second finger of the user simultaneously pressing another one physiological signal sensing unit 12 via the touch plane 123, the lighting element 121 emits a second detection light to the second finger for making the light sensing element 122 receive a second reflective light from the second finger, such that the main controller 111 receives a second sensing signal from the light sensing element 122, so as to utilize the physiological parameter calculating unit 1111 to calculate the user's physiological parameters including blood pressure and blood oxygen saturation level after applying the physiological parameter calculating process to the second sensing signal. As such, the user's physiological parameters including heartbeat, heart rate, blood pressure, and blood oxygen saturation level are all measured by using the two physiological signal sensing units.

As FIG. 2 and FIG. 3 show, the electronic device is a desk computer, and the main controller 111 transmits a physiological parameter data comprising the heartbeat, the heart rate, the blood pressure, and the blood oxygen saturation level to the electronic device 2 through a wired communication unit 117, such that the electronic device 2 shows the heartbeat, the heart rate, the blood pressure, and/or the blood oxygen saturation level on a display screen of a display device 22. Herein, it needs to further explain that, the electronic device 2 is not limited to be a desk computer. In a practicable embodiment, the electronic device 2 can also be a laptop computer, a tablet computer, a smart phone, or a smart TV. Furthermore, it is able to make the electronic device 2 has a physiological parameter managing program for achieving a data management of the physiological parameter data. The data management comprises: data storing, data transmitting, displaying the physiological parameter data by a form of graphs or diagrams, and/or displaying the physiological parameter data by a form of numeric values.

It is worth further explaining that, the lighting element 121 is not limited to be a light-emitting diode (LED), but can also be a quantum dot light emitting diode (QD-LED), a mini LED, a micro LED, or an organic light-emitting diode (OLED). As such, the first detection light (or the second detection light) radiated from the lighting element 121 can be controlled to be a single-wavelength light or a multi-wavelength light. In other words, in case of taking a LED component as the lighting element 121, the detection light emitted by the LED component can be a single-wavelength light or a multi-wavelength light comprises a green light (400-600 nm), a red light (600-800 nm) and an infrared light (800-1000 nm). Moreover, when taking an OLED component as the lighting element 121, the detection light emitted by the OLED component can be a single-wavelength light or a multi-wavelength light comprises a green light, a red light and an infrared light. Moreover, for sensing the single-wavelength light and/or the multi-wavelength light, the light sensing element 122 can be a single point photo sensor, a matrix photo sensor, a one-channel image sensor, or a multi-channel image sensor.

As described in more detail below, both the first sensing signal and the second sensing signal are a photoplethysmography (PPG) signal, and the physiological parameter calculating unit 111 applies at least one signal process to the first sensing signal and the second sensing signal before executing the physiological parameter calculating process. For calculating the user's physiological parameters including the heartbeat, heart rate, the blood pressure, and the blood oxygen saturation level, the forgoing signal process can be a signal conversion process for converting a time-domain signal (i.e., the sensing signal) to a frequency-domain signal. The signal conversion process can be selected from the group consisting of fast Fourier transforming (FFT) process, discrete Fourier transforming (DFT) process and short-time Fourier transforming (STFT) process. On the other hand, the signal process can also be a signal analyzing process that is selected from the group consisting of signal process using singular spectrum analysis (SSA) algorithm and signal process using normalized least mean square (NLMS) algorithm.

### Second Embodiment

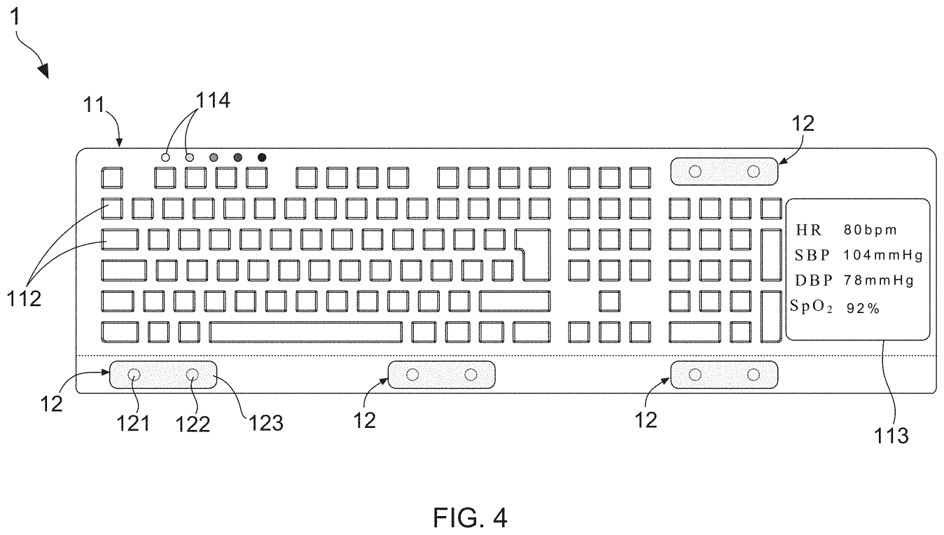

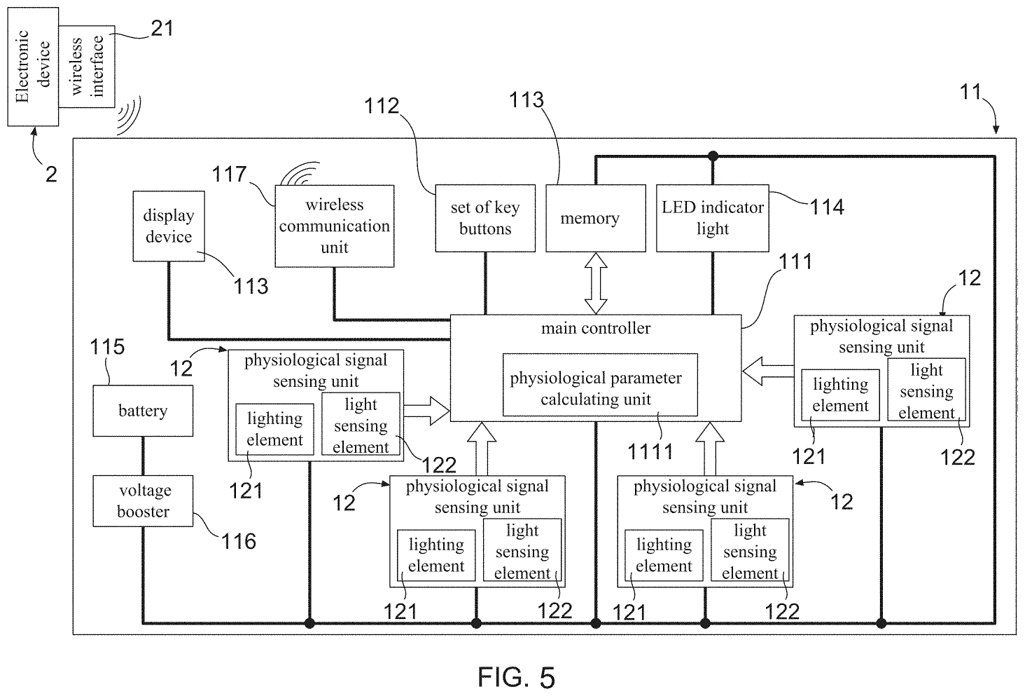

With reference to FIG. 4, there is provided a top-view diagram of a keyboard device having functionality of physiological parameter measurement according to the present invention. Moreover, FIG. 5 shows a block diagram of the keyboard device having functionality of physiological parameter measurement according to the present invention. After comparing FIG. 4 with FIG. 1 as well as comparing FIG. 5 with FIG. 2, it is easy to find that, the second embodiment of the keyboard device 1 of the present invention further comprises a display device 1D. The display device 1D is coupled to the main controller 111, and a display screen of the display device 1D is exposed out of the keyboard main body 11. By such arrangement, after the first finger and/or the second press on touch planes 123, the main controller 111 is able to show an indicating information, through the display screen of the display device 1D, for informing the user that a physiological parameter measurement is beginning to be executed. Moreover, the main controller 111 is able to shows the heartbeat, the heart rate, the blood pressure, and/or the blood oxygen saturation level on the display screen of the display device 1D after the physiological parameter measurement is completed.

### Third Embodiment

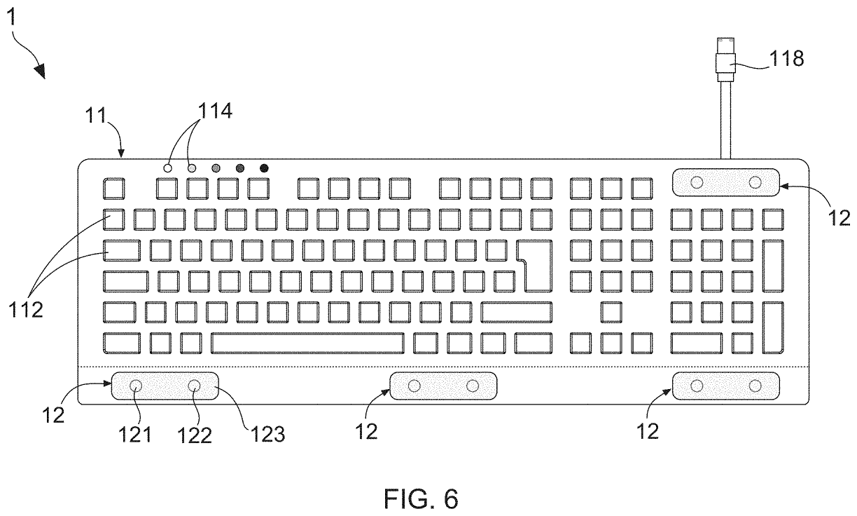

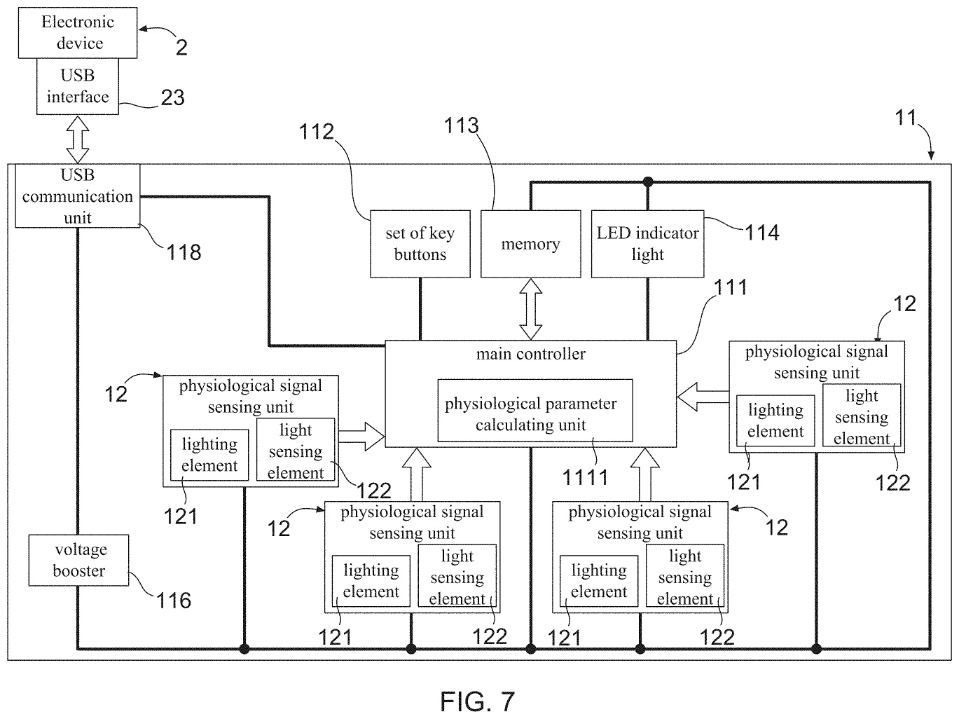

With reference to FIG. 6, there is provided a top-view diagram of a keyboard device having functionality of physiological parameter measurement according to the present invention. Moreover, FIG. 7 shows a block diagram of the keyboard device having functionality of physiological parameter measurement according to the present invention. After comparing FIG. 6 with FIG. 1 as well as comparing FIG. 7 with FIG. 2, it is easy to find that, the third embodiment of the keyboard device 1 of the present invention also comprises a keyboard main body 11 and at least two physiological signal sensing units 12. In third embodiment, however, the keyboard main body 11 is a wired computer keyboard, and principally comprises a main controller 111, a set 112 of key buttons, a memory 113, at least one LED indicator light 114, a voltage booster 116, and a USB communication unit 118. Herein, wired computer keyboard is a well-known inputting device, which communicates with a USB interface 23 of an electronic device 2. For this reason, it is not needed to introduce the basic constituting elements of the wired computer keyboard.

Therefore, above descriptions have introduced the embodiments of the keyboard device having functionality of physiological parameter measurement of the present invention clearly and completely. However, any modification to the present invention made by a person skilled in the art does not depart from the protection scope defined by the appended claims.

What is claimed is:

1. A keyboard device having functionality of physiological parameter measurement, comprising:
a keyboard main body having a main controller and a plurality of key buttons, wherein the main controller is provided with a physiological parameter calculating unit therein; and
at least two physiological signal sensing units, being disposed in the keyboard main body; wherein each of the at least two physiological signal sensing units comprises at least one lighting element and at least one light sensing element, and further comprising a touch plane that is exposed out of a surface of the keyboard main body;
wherein in case of a first finger of a user pressing one physiological signal sensing unit via the touch plane, the lighting element emitting a first detection light to the first finger for making the light sensing element receive a first reflective light from the first finger, such that the main controller receives a first sensing signal from the light sensing element, so as to utilize the physiological parameter calculating unit to calculate at least one first physiological parameter after applying a physiological parameter calculating process to the first sensing signal;
wherein in case of a second finger of the user simultaneously pressing another one physiological signal sensing unit via the touch plane, the lighting element emitting a second detection light to the second finger for making the light sensing element receive a second reflective light from the second finger, such that the main controller receives a second sensing signal from the light sensing element, so as to utilize the physiological parameter calculating unit to calculate at least one second physiological parameter after applying the physiological parameter calculating process to the second sensing signal;
wherein the at least one first physiological parameter and the at least one second physiological parameter comprises heartbeat, heart rate, blood pressure, and blood oxygen saturation level.

2. The keyboard device according to claim 1, wherein the keyboard main body further comprises a display device that is coupled to the main controller, and a display screen of the display device being exposed out of the keyboard main body, such that the main controller shows an indicating information, through the display screen, for informing the user that a physiological parameter measurement is beginning to be executed.

3. The keyboard device according to claim 2, wherein the main controller shows the heartbeat, the heart rate, the blood pressure, and/or the blood oxygen saturation level on the display screen after the physiological parameter measurement is completed.

4. The keyboard device according to claim 1, wherein the main controller transmits a physiological parameter data comprising the heartbeat, the heart rate, the blood pressure, and the blood oxygen saturation level to an electronic device through a wired communication interface or a wireless communication interface, such that the electronic device shows the heartbeat, the heart rate, the blood pressure, and/or the blood oxygen saturation level on a display screen thereof.

5. The keyboard device according to claim 4, wherein the electronic device is selected from the group consisting of desk computer, laptop computer, tablet computer, smart phone, and smart TV.

6. The keyboard device according to claim 5, wherein the electronic device has a physiological parameter managing program for achieving a data management of the physiological parameter data, and the data management being selected from the group consisting of data storing, data transmitting, displaying the physiological parameter data by a form of graphs or diagrams, and displaying the physiological parameter data by a form of numeric values.

7. The keyboard device according to claim 5, wherein the lighting element is selected from the group consisting of light-emitting diode (LED), quantum dot light emitting diode (QD-LED), mini LED, micro LED, and organic light-emitting diode (OLED).

8. The keyboard device according to claim 1, wherein both the first detection light and the second detection light are a single-wavelength light or a multi-wavelength light.

9. The keyboard device according to claim 1, wherein the light sensing element is selected from the group consisting of single point photo sensor, matrix photo sensor, one-channel image sensor, and multi-channel image sensor.

10. The keyboard device according to claim 1, wherein both the first sensing signal and the second sensing signal are a photoplethysmography (PPG) signal, and the physiological parameter calculating unit applying at least one signal process to the first sensing signal and the second sensing signal before executing the physiological parameter calculating process.

11. The keyboard device according to claim 10, wherein the signal process is selected from the group consisting of fast Fourier transforming (FFT) process, discrete Fourier transforming (DFT) process and short-time Fourier transforming (STFT) process.

12. The keyboard device according to claim 10, wherein the signal process is selected from the group consisting of signal process using singular spectrum analysis (SSA) algorithm and signal process using normalized least mean square (NLMS) algorithm.
